(19) United States
(12) Patent Application Publication
(10) Pub. No.: US 2009/0017118 A1
(43) Pub. Date: Jan. 15, 2009

(54) COATED TABLETS, THEIR METHODS OF PREPARATION, AND RELATED USES

(75) Inventors: Peter B. Samuelsen, Gentofte (DK); Leif Knudsen, Grenaa (DK); Kern Lystrup, Veerlose (DK)

Correspondence Address: MARSHALL, GERSTEIN & BORUN LLP, 233 S. WACKER DRIVE, SUITE 6300, SEARS TOWER, CHICAGO, IL, 60606, US

(73) Assignee: SPORTSCOM DANMARK APS, Charlottenlund (DK)

(21) Appl. No.: 12/278,678

(22) PCT Filed: Feb. 9, 2007
(86) PCT No.: PCT/DK2007/000068

§ 371 Date: Sep. 24, 2008

(30) Foreign Application Priority Data
Feb. 10, 2006 (DK) PA200600194

(51) Int. Cl. A61K 9/32

(52) U.S. Cl. 424/482

(57) ABSTRACT

The present invention relates to a tablet comprising a tabletted core comprising a triglyceride granulate, and an enteric coating surrounding said tabletted core. The invention particularly relates to a tablet wherein the tabletted core contains esterified omega-3 fatty acids such as eicosapentaenoic acid and/or docosahexaenoic acid.

This application is a §371 national filing of International (PCT) Application No. PCT/DK2007/000068 filed Feb. 9, 2007, which claims the benefit of priority of Denmark Patent Application No. PA2006 00194 filed Feb. 10, 2006. Each of the foregoing applications are incorporated herein by reference.

### TECHNICAL FIELD OF THE INVENTION

The present invention relates to a tablet comprising a tabletted core comprising a triglyceride granulate, and an enteric coating surrounding said tabletted core. The invention particularly relates to a tablet wherein the tabletted core contains esterified omega-3 fatty acids such as eicosapentaenoic acid and/or docosahexaenoic acid.

### BACKGROUND OF THE INVENTION

It is generally recognised that triglycerides, such as fish oil, containing esterified omega-3 acids are important for a healthy human diet.

Some experts believe that taking fish oil (in any form) can help regulate cholesterol in the body, because fish oil has high levels of omega-3 fatty acids. The regulation occurs through effects of the EPA and DHA constituents on Peroxisome proliferator-activated receptor alpha (PPARα). Besides cholesterol regulation, benefits include anti-inflammatory properties and positive effects on body composition.

In order to receive the required amount of fish oil every day, fish oil in liquid form may be taken besides the vitamin and mineral pills. The liquid fish oil may e.g. be taken in a capsule form. However, some people who ingest large amounts of fish oil each day will experience gastrointestinal upset and burp up a “fishy” smell even hours after the fish oil is taken. Many people may therefore refrain from supplementing their diet with fish oil capsules.

Other drawbacks of vitamin and mineral pills and fish oil capsules are that several pills and capsules must be taken each day. Capsules of highly concentrated fish oil are produced in order to reduce the volume, and in turn reduce the daily number of capsules required.

### PRIOR ART

EP-A-0 276 772 describes a process for preparing a microdispersed, pulverulent or aqueous fish oil preparation with a high concentration of the active substances of the fish oil, in particular EPA and DHA. This preparation of fish oil may result in a reduction of the bad smell and taste of fish oil and is used in baby food and dry-powdered milk as well as supplements in bakery and other nutritional food products.

EP-A-1 155 620 discloses a tablet comprising vitamins, minerals, and a fish oil granulate. A problem associated with producing a tablet of such kind is that the microdispersed fish oil may not be subjected to an excessive pressure during the tablet manufacturing. A too high compression pressure of the mixture of tablet ingredients including the fish oil granulate will cause the microdispersed grains to burst and fish oil will leak out into the composition mixture. This will have an adverse effect of the tablet and may cause the tablets to disintegrate during the pressing. Moreover, the fish oil will rapidly become harsh and the tablet will deteriorate quickly, besides having an unpleasant smell of fish oil.

Another problem associated with microdispersed fish oil granulate in a tablet is that although the smell is reduced compared to fish oil in liquid form, the tablet will nevertheless have a “fishy” odour.

### SUMMARY OF THE INVENTION

An object of the present invention is to provide improved triglyceride tablets or capsules, which solve the problems of the prior art products.

Yet an object of the present invention is to provide triglyceride tablets, which are easier and less troublesome to ingest than prior art fish oil tablets and capsules.

The present invention is based on the surprising discovery that when ingested, tablets containing a triglyceride granulate and an enteric coating give rise to a much high bioavailability of the fatty acids of the triglyceride than tablets without an enteric coating.

Without being bound by theory, it is believed that the enteric coating delays the release of the triglycerides until the tablet arrives in the intestines. When released in the intestines, the triglyceride is believed to be microdispersed as in the triglyceride granulate, thus having a high effective surface area through which the lipases of the intestines can degrade the triglycerides to glycerol and fatty acids.

Thus, one aspect of the invention relates to a tablet comprising
a) a tabletted core containing
   a triglyceride granulate comprising triglycerides containing one or more esterified omega-3 fatty acids, and
   excipients, and
b) an enteric coating surrounding the tabletted core, said enteric coating comprising a coating material.

Another aspect of the present invention relates to a method of preparing a tablet comprising a coated, tabletted core, the method comprising the steps of
i) providing a tabletted core containing
   a triglyceride granulate comprising triglycerides containing one or more esterified omega-3 fatty acids,
   a nutricient,
   excipients, and
ii) coating said tabletted core with an enteric coating material.

Yet an aspect of the invention relates to various uses of the tablet as well as the coating and the triglyceride granulate.

### BRIEF DESCRIPTION OF THE FIGURE

FIG. 1 shows the bioavailability of DHA and EPA in the blood stream of minipigs after administration of tablets containing triglycerides comprising esterified DHA and EPA.

The present invention will now be described in more detail in the following.

### DETAILED DESCRIPTION OF THE INVENTION

The tablet of the present invention contains a tabletted core and an enteric coating.

The tabletted core comprises a triglyceride granulate, and excipients. It is preferred that the triglyceride granulate and the excipients as well as the parameters of the tabletting process are selected so as to provide a coherent and robust tabletted core which does not disintegrate during post-processing and coating of the tabletted core. Preferably, the granules of the triglyceride granulate are pressed together with the other components of the tabletted core. The tablet additionally contains an enteric coating which preferably surrounds the tabletted core and acts as a barrier layer between the surroundings and the tabletted core.

In the context of the present invention, the term “triglyceride granulate” relates to a granulate comprising a triglyceride and one or more granulate additives. A number of different triglyceride granulates are known to the person skilled in the art, for example the ones disclosed in EP-A-0 276 772, the contents of which are incorporated herein by reference. The triglycerides of the triglyceride granulate are preferably microdispersed in the triglyceride granulate.

Importantly, at least some of the triglycerides of the triglyceride granulate comprise one or more esterified omega-3 fatty acids. While other triglyceride sources may be used, fish oil or vegetable oil is presently preferred. Thus, in a preferred embodiment of the invention, the triglyceride granulate is a fish oil granulate.

Omega-3 fatty acids are a family of polyunsaturated fatty acids which have in common a carbon-carbon double bond in the ω-3 position. Useful omega-3 fatty acids are e.g. alpha-linolenic acid (ALA), eicosapentaenoic acid (EPA), and docosahexaenoic acid (DHA). In some embodiments of the invention, the esterified omega-3 fatty acids of the triglyceride comprises esterified EPA and/or esterified DHA.

In the context of the present invention, the term “and/or” used in the context “X and/or Y” should be interpreted as “X”, or “Y”, or “X and Y”.

Thus, the triglyceride may comprise esterified EPA, it may comprise esterified DHA, and it may comprise both esterified DHA and esterified EPA.

In preferred embodiments of invention, at least 15% by mol of the esterified omega-3 fatty acid is either EPA or DHA, such as at least 25%, preferably at least 40%, such as at least 50% and even more preferably at least 60%, such as at least 75% by mol.

In preferred embodiments of the invention, the tabletted core additionally comprises one or more vitamins and/or one or more minerals.

The one or more vitamins typically include at least one vitamin selected from the group consisting of vitamin A, beta-carotene, vitamin B₁, vitamin B₂, vitamin B₃, vitamin B₅, vitamin B₆, vitamin B₇, vitamin B₉, vitamin B₁₂, vitamin C, vitamin D₃, vitamin E, vitamin K, pantothenic acid, folic acid, biotin, and mixtures thereof.

In some embodiments of the invention, the tablet contains one or more of the above mentioned vitamins in an amount in the range of ±25% of the Recommended Dietary Allowance (RDA) (see Table 1) of the one or more vitamins, preferably in the range of ±15% of the RDA, and even more preferably in the range of ±10% of the RDA, such as in the range of ±5% of the RDA.

TABLE 1
Recommended Dietary Allowance for various vitamins.

| | Recommended | |
|---|---|---|
| Vitamin | Dietary Allowance | |
| Vitamin A | 900 | μg |
| Vitamin B₁ | 1.2 | mg |
| Vitamin B₂ | 1.3 | mg |
| Vitamin B₃ | 16.0 | mg |
| Vitamin B₅ | 5.0 | mg |
| Vitamin B₆ | 1.3-1.7 | mg |
| Vitamin B₇ | 30.0 | μg |
| Vitamin B₉ | 400 | μg |
| Vitamin B₁₂ | 2.4 | μg |
| Vitamin C | 90.0 | mg |
| Vitamin D | 5.0 μg-10 μg | |
| Vitamin E | 15.0 | mg |
| Vitamin K | 120 | μg |

The one or more minerals typically include at least one mineral selected from the group consisting of a calcium mineral, a magnesium mineral, a zinc mineral, an iron mineral, an iodine mineral, a selenium mineral, a chromium mineral, a manganese mineral, a molybdenum mineral, and mixtures thereof.

In the context of the present invention the term “enteric coating” relates to a coating which is resistant to the acidic environment of the stomach but dissolves or disintegrates when it reaches the intestines.

In some preferred embodiments, the coating is a pH-sensitive enteric coating, that is, a coating is stable at acidic pH, but breaks down rapidly at neutral or basic pH.

Advantageously, the triglyceride granulate size and composition, the excipients as well as the materials and amount of the coating may be so selected that the resulting tablet releases at most 25% of the triglyceride during the first two hours of testing when performing the dissolution test for solid dosage forms in simulated gastric fluid using the Ph. Eur. paddle method at 100 rpm, and preferably the tablet releases at most 20% of the triglyceride during the first two hours, such as at most 15% of the triglyceride during the first two hours, and even more preferably at most 10% of the triglyceride during the first two hours.

In some embodiments of the invention, the tablet releases at least 10% of the triglyceride during the first two hours of testing when performing the dissolution test for solid dosage forms in simulated intestinal fluid using Ph. Eur. paddle method at 100 rpm. Preferably the tablet releases at least 20% of the triglyceride during the first two hours, preferably at least 40% of the triglyceride during the first two hours, and even more preferably at least 50% of the triglyceride during the first two hours.

The dissolution test as well as the paddle method are described in the European Pharmacopoeia (Ph. Eur.) 3rd edition, ISBN: 92-871-2991-6, 1997, the contents of which are incorporated herein by reference for all purposes.

For clarity sake, the composition of simulated gastric fluid (SGF) and simulated intestinal fluid (SIF), although well known in the art as standard solutions, are set forth below:

SGF (USP Simulated Gastric Fluid without pepsin) composition:

### HCl qs pH 1.2

### NaCl 0.2%

water qs 1000 mL
SIF (USP Simulated Intestinal Fluid without pancreatin) composition:

### KH₂PO₄ 6.8 g

### NaOH qs pH 6.8

water qs 1000 mL

In some embodiments of the invention the coating material comprises, or essentially consists of, a pharmaceutically acceptable acid-resistant polymer.

In some embodiments of the invention, the coating material has a solubility at 25° C. of at most 5 g coating material per 100 g acidic aqueous solution, such as at most 2 g coating material per 100 g acidic aqueous solution, preferably at most 5 g coating material per 100 g acidic aqueous solution, such as at most 10 g coating material per 100 g acidic aqueous solution, and even more preferably at solubility of at most 15 g coating material per 100 g acidic aqueous solution,

said acid aqueous solution consisting of 1 mM HCl dissolved in demineralised water.

In some embodiment of the invention, the coating material has a solubility at 25° C. of at least 0.5 g coating material per 100 g basic aqueous solution, such as at least 1 g coating material per 100 g basic aqueous solution, preferably at least 5 g coating material per 100 g basic aqueous solution, such as at least 10 g coating material per 100 g basic aqueous solution, and even more preferably a solubility of at least 15 g coating material per 100 g basic aqueous solution,

said basic aqueous solution consisting of 1 mM NaOH dissolved in demineralised water.

The coating material may e.g. comprise at least one material selected from the group consisting of acid-resistant acrylic polymer, acid-resistant methacrylic polymer, modified cellulose, methacrylic acid copolymers, cellulose acetate (and its succinate and phthalate version), styrol maleic acid co-polymers, polymethacrylic acid/acrylic acid copolymer, hydroxypropyl methyl cellulose phthalate, polyvinyl acetate phthalate, hydroxyethyl ethyl cellulose phthalate, hydroxypropyl methyl cellulose acetate succinate, cellulose acetate tetrahydrophtalate, acrylic resin, timellitate, shellac, and combinations thereof.

In a preferred embodiment of the invention, the enteric coating comprises shellac, e.g. bleached shellac or bleached, dewaxed shellac.

The coating material may e.g. comprise one or more pharmaceutically acceptable coating additives. For example, an anti-sticking agent, such as talc, may be added to the coating to avoid stickiness of the coated, tabletted cores. Similarly, a plasticizer such as triethylcitrate can improve the characteristics of the coating.

In some embodiment of the invention, the smallest thickness of the coating is at least 5 micron, such as at least 20 micron, preferably at least 50 micron such as at least 100 micron, and even more preferably at least 200 micron such as at least 400 micron.

In some embodiments of the invention, the thickness of the coating is in the range of 5 micron-5 mm, such as 10 micron to 1 mm, preferably in the range of 25 micron-500 micron, such as in the range of 50 micron-250 micron, and even more preferably in the range of 75 microns-150 micron.

An additional advantage of the enteric coating is that is reduces the “fishy” smell which typically is associated with the uncoated, tabletted cores.

Yet an advantage of the invention is that it improves the biological absorption of omega-3 fatty acids from triglycerides such as fish oil. This means that a reduced amount of fish oil used according to the present invention will have the same biological effect as a much larger amount of fish oil used according to the prior art.

The triglyceride granulate typically has an average granule size in the range of 1 micron-2 mm, such as 10 micron to 1.5 mm, preferably in the range of 25 micron-1 mm, such as in the range of 50 micron-500 micron, and even more preferably in the range of 75 microns-250 micron.

The granule size of a triglyceride granule is measured as the length of the longest dimension of the granule.

The triglyceride granulate normally comprises in the range of 5-80% triglyceride by weight, such as 10-70% triglyceride by weight, preferably in the range of 15-60% triglyceride by weight, and even more preferably in the range of 20-50% triglyceride by weight, such as in the range of 25-45% triglyceride by weight.

The triglyceride granulate may for example comprise in the range of 2-75% esterified omega-3 fatty acids by weight, such as 5-70% esterified omega-3 fatty acids by weight, preferably in the range of 10-60% esterified omega-3 fatty acids by weight, and even more preferably in the range of 15-45% esterified omega-3 fatty acids by weight, such as in the range of 20-40% esterified omega-3 fatty acids by weight.

The triglyceride granulate typically comprises one or more granulate additives. A number of useful granulate additives is known to the person skilled in the art, see e.g. EP-A-0 276 772, the contents of which are incorporate herein by reference.

The granulate additives are typically relatively inert materials that are able to bind or contain the triglyceride. For example, at least one granulate additive may be selected from the group consisting of starches, microcrystalline cellulose (crystalline cellulose in other terminology), alpha lactose, dextrin, mannitol, chitosan, or combinations thereof.

The tabletted core normally comprises in the range of 20-85% triglyceride granulate by weight, such as 25-75% triglyceride granulate by weight, preferably in the range of 30-70% triglyceride granulate by weight, and even more preferably in the range of 35-65% triglyceride granulate by weight, such as in the range of 40-60% triglyceride granulate by weight.

The tabletted core may e.g. comprise in the range of 10-70% triglyceride by weight, such as 15-65% triglyceride by weight, preferably in the range of 20-60% triglyceride by weight, and even more preferably in the range of 25-55% triglyceride by weight, such as in the range of 30-50% triglyceride by weight.

The tabletted core normally comprises one or more excipients.

A number of useful excipients for the production of tablets are well known to the person skilled in the art, and may e.g. be found in the Handbook of Pharmaceutical Excipients, edited by Raymond Rowe et al., Pharmaceutical Press London, 4rd edition.

The tabletted core may e.g. comprise excipients such as magnesium stearate, plasticizers (e.g. triethylcitrate) and/or or anti-sticking agents (e.g. talc).

Preferably, the tablet comprises in the range of 0.1-50% esterified omega-3 fatty acids by weight, such as 0.5-25% esterified omega-3 fatty acids by weight, preferably in the range of 1-20% esterified omega-3 fatty acids by weight, and even more preferably in the range of 2-18% esterified omega-3 fatty acids by weight, such as in the range of 5-15% esterified omega-3 fatty acids by weight.

The tablets typically contains in the range of 5-750 mg esterified omega-3 fatty acids, such as in the range of 10-500 mg esterified omega-3 fatty acids, preferably in the range of in the range of 25-250 mg esterified omega-3 fatty acids, and even more preferably in the range of in the range of 50-130 mg esterified omega-3 fatty acids.

In some embodiments of the invention, one tablet contains the RDA of vitamins, minerals and omega-3 fatty acids.

In the context of the present invention, the RDA for omega-3 fatty acids (esterified and free fatty acids) is 480 mg.

Thus, in some embodiments of the invention, the tablet contains esterified omege-3 fatty acids in an amount in the range of ±25% of the RDA of omege-3 fatty acids, preferably in the range of ±15% of the RDA, and even more preferably in the range of ±10% of the RDA, such as in the range of ±5% of the RDA.

In some embodiments of the invention, one tablet contains esterified omege-3 fatty acids in an amount in the range of ±25% of the RDA of omege-3 fatty acids, preferably in the range of ±15% of the RDA, and even more preferably in the range of ±10% of the RDA, such as in the range of ±5% of the RDA.

In some embodiments of the invention, one tablet contains one or more of the above mentioned vitamins in an amount in the range of ±25% of the Recommended Dietary Allowance (RDA) of the one or more vitamins, preferably in the range of ±15% of the RDA, and even more preferably in the range of ±10% of the RDA, such as in the range of ±5% of the RDA.

In some embodiments of the invention, one tablet contains one or more of the above mentioned minerals in an amount in the range of ±25% of the Recommended Dietary Allowance (RDA) of the one or more minerals, preferably in the range of ±15% of the RDA, and even more preferably in the range of ±10% of the RDA, such as in the range of ±5% of the RDA.

It is also envisioned that two tablets in combination may contain the RDA of vitamins, minerals and omega-3 fatty acids.

In some embodiments of the invention, one tablet contains esterified omege-3 fatty acids in an amount in the range of ±25% of 0.5*RDA of omege-3 fatty acids, preferably in the range of ±15% of 0.5*RDA, and even more preferably in the range of ±10% of 0.5*RDA, such as in the range of ±5% of 0.5*RDA.

In some embodiments of the invention, one tablet contains one or more of the above mentioned vitamins in an amount in the range of ±25% of 0.5*RDA of the one or more vitamins, preferably in the range of ±15% of 0.5*RDA, and even more preferably in the range of ±10% of 0.5*RDA, such as in the range of ±5% of 0.5*RDA.

In some embodiments of the invention, one tablet contains one or more of the above mentioned minerals in an amount in the range of ±25% of 0.5*RDA of the one or more minerals, preferably in the range of ±15% of 0.5*RDA, and even more preferably in the range of ±10% of 0.5*RDA, such as in the range of ±5% of 0.5*RDA.

It is furthermore envisioned that three tablets in combination may contain the RDA of vitamins, minerals and omega-3 fatty acids.

In some embodiments of the invention, one tablet contains esterified omege-3 fatty acids in an amount in the range of ±25% of 0.33*RDA of omege-3 fatty acids, preferably in the range of ±15% of 0.33*RDA, and even more preferably in the range of ±10% of 0.33*RDA, such as in the range of ±5% of 0.33*RDA.

In some embodiments of the invention, one tablet contains one or more of the above mentioned vitamins in an amount in the range of ±25% of 0.33*RDA of the one or more vitamins, preferably in the range of ±15% of 0.33*RDA, and even more preferably in the range of ±10% of 0.33*RDA, such as in the range of ±5% of 0.33*RDA.

In some embodiments of the invention, one tablet contains one or more of the above mentioned minerals in an amount in the range of ±25% of 0.33*RDA of the one or more minerals, preferably in the range of ±15% of 0.33*RDA, and even more preferably in the range of ±10% of 0.33*RDA, such as in the range of ±5% of 0.33*RDA.

Additionally, it is envisioned that four tablets in combination may contain the RDA of vitamins, minerals and omega-3 fatty acids.

In some embodiments of the invention, one tablet contains esterified omege-3 fatty acids in an amount in the range of ±25% of 0.25*RDA of omege-3 fatty acids, preferably in the range of ±15% of 0.25*RDA, and even more preferably in the range of ±10% of 0.25*RDA, such as in the range of ±5% of 0.25*RDA.

In some embodiments of the invention, one tablet contains one or more of the above mentioned vitamins in an amount in the range of ±25% of 0.25*RDA of the one or more vitamins, preferably in the range of ±15% of 0.25*RDA, and even more preferably in the range of ±10% of 0.25*RDA, such as in the range of ±5% of 0.25*RDA.

In some embodiments of the invention, one tablet contains one or more of the above mentioned minerals in an amount in the range of ±25% of 0.25*RDA of the one or more minerals, preferably in the range of ±15% of 0.25*RDA, and even more preferably in the range of ±10% of 0.25*RDA, such as in the range of ±5% of 0.25*RDA.

The weight of the tabletted core relative to the total weight of the tablet may be varied according to the requirement of the consumers. Thus, the tablet may e.g. comprise in the range of 1-99% tabletted core by weight, such as 10-90% tabletted core by weight, preferably in the range of 20-80% tabletted core by weight such as 25-75% tabletted core by weight, and even more preferably in the range of 30-70% tabletted core by weight, such as in the range of 40-60% tabletted core by weight.

Preferably, the tablet comprises in the range of 1-75% coating by weight, such as 10-65% coating by weight, preferably in the range of 15-60% coating by weight such as 20-55% coating by weight, and even more preferably in the range of 25-50% coating by weight, such as in the range of 30-45% coating by weight.

Typically, the tablet has a weight in the range of 100 mg-5 g, such as in the range of 250 mg-2.5 g, preferably in the range of 500 mg-2 g, and even more preferably in the range of 750 mg-1.5 g.

In preferred embodiments of the invention, the tablet comprises
a) a tabletted core comprising
   a triglyceride granulate in an amount of 40-60% by weight of the tabletted core, said triglyceride granulate comprising in the range of 20-40% esterified omega-3 fatty acids by weight of the triglyceride granulate.
   and one or more granulate additives,
said tabletted core comprising in the range of 30-70% by weight of the tablet, and
b) an enteric coating surrounding the tabletted core,
   said coating comprising in the range of 30-70% by weight of the tablet,
   said tablet having a weight in the range of 0.5 g-2 g.

Yet an aspect of the invention, relates to a method of preparing a tablet comprising a coated, tabletted core, the method comprising the steps of
i) providing a tabletted core containing
   a triglyceride granulate comprising triglycerides containing one or more esterified omega-3 fatty acids,
   excipients, and
ii) coating said tabletted core with an enteric coating material.

The coating may e.g. be applied to the tabletted core as an aqueous film of a coating solution comprising the coating material.

A further aspect of the invention relates to the use of an enteric coating as defined herein for increasing the bioavailability of omega-3 fatty acids from a triglyceride granulate comprising triglyceride esters of said omega-3 fatty acids.

Yet an aspect of the invention relates to the use of an enteric coating as defined herein for increasing the biological absorption of omega-3 fatty acids from triglyceride granulate, said triglyceride granulate containing esterified omega-3 fatty acids.

Additional aspects of the invention relates to medical uses of the tabletted core and the coating material, e.g.:

Use of a tabletted core as defined herein and one or more coating materials as defined herein for the manufacture of a medicament for treatment or prevention of depression.

Use of a tabletted core as defined herein and one or more coating materials as defined herein for the manufacture of a medicament for treatment or prevention of cancer.

Use of a tabletted core as defined herein and one or more coating materials as defined herein for the manufacture of a medicament for treatment or prevention of schizophrenia.

Use of a tabletted core as defined herein and one or more coating materials as defined herein for the manufacture of a medicament for treatment or prevention of Alzheimer's disease.

Use of a tabletted core as defined herein and one or more coating materials as defined herein for the manufacture of a medicament for treatment or prevention of cardiovascular diseases.

Use of a tabletted core as defined herein and one or more coating materials as defined herein for the manufacture of a medicament for treatment or prevention of Arthritis.

Use of a tabletted core as defined herein and one or more coating materials as defined herein for the manufacture of a medicament for treatment or prevention of osteoporosis.

It is particularly preferred that the coating materials are used in the manufacture to provide an enteric coating surrounding the tabletted core.

It should be noted that embodiments and features described in the context of one of the aspects of the present invention also apply to the other aspects of the invention.

All patent and non-patent references cited in the present application are hereby incorporated by reference in their entirety.

The invention will now be described in further details in the following non-limiting examples.

### EXAMPLE

A composition with ingredients as specified in Table 1 was mixed and pressed to a “green block” in a tablet forming tool in a conventional tablet pressing machine using a pressing pressure which is somewhat lower than the normal pressure. The thereby produced “green block” was then coated in a coating apparatus with a biodegradable coating. The coating is an aqueous film coating shellac, such as the FDA approved product CertiSeal FC-300™.

TABLE 2

| | Amount | |
|---|---|---|
| Ingredients | | |
| C-vitamin | 12 | mg |
| E-vitamin | 2.0 | mg |
| Calcium | 80 | mg |
| Magnesium | 30 | mg |
| Niacin | 3.6 | mg |
| Vitamin B₆ | 0.4 | mg |
| Pantothenic acid | 1.2 | mg |
| Vitamin B₁ | 0.28 | mg |
| Vitamin B₂ | 0.32 | mg |
| Vitamin B₁₂ | 0.20 | μg |
| Zinc | 3.0 | mg |
| Iron | 2.8 | mg |
| Vitamin A | 160 | μg |
| Vitamin D₃ | 1.0 | μg |
| Folic acid | 40 | μg |
| Biotin | 30 | μg |
| Iodine | 30 | μg |
| Selenium | 10.0 | μg |
| Chromium | 10.0 | μg |
| Omega-3 fatty acids (DHA and EPA) | 44 | mg |
| Other ingredients | | |
| Starch Ph. Eur. | 18.4 | mg |
| Microcrystalinic cellulose Ph. Eur. | 132 | mg |
| Polyvidon Ph. Eur. | 19.2 | mg |
| Talcum Ph. Eur. | 6.4 | mg |
| Magnesium stearate Ph. Eur. | 3.2 | mg |

Hereby, a tablet is provided for nutritional supplement including active ingredients including microdispersed granular oil, such as fish oil proportionally selected in accordance with a recommended daily allowance. This basis composition of the tablet may be altered in accordance with customer specific demands.

In a first experiment, three types of fish oil tablets were produced:
A tablet without any coating,
A tablet with an aqueous based shellac coating with a coating thickness of 203 g/kg,
A tablet with an aqueous based shellac coating with a coating thickness of 406 g/kg, i.e. a double coating.

The shellac coating is natural lactose resin, plasticizers or other adjuncts.

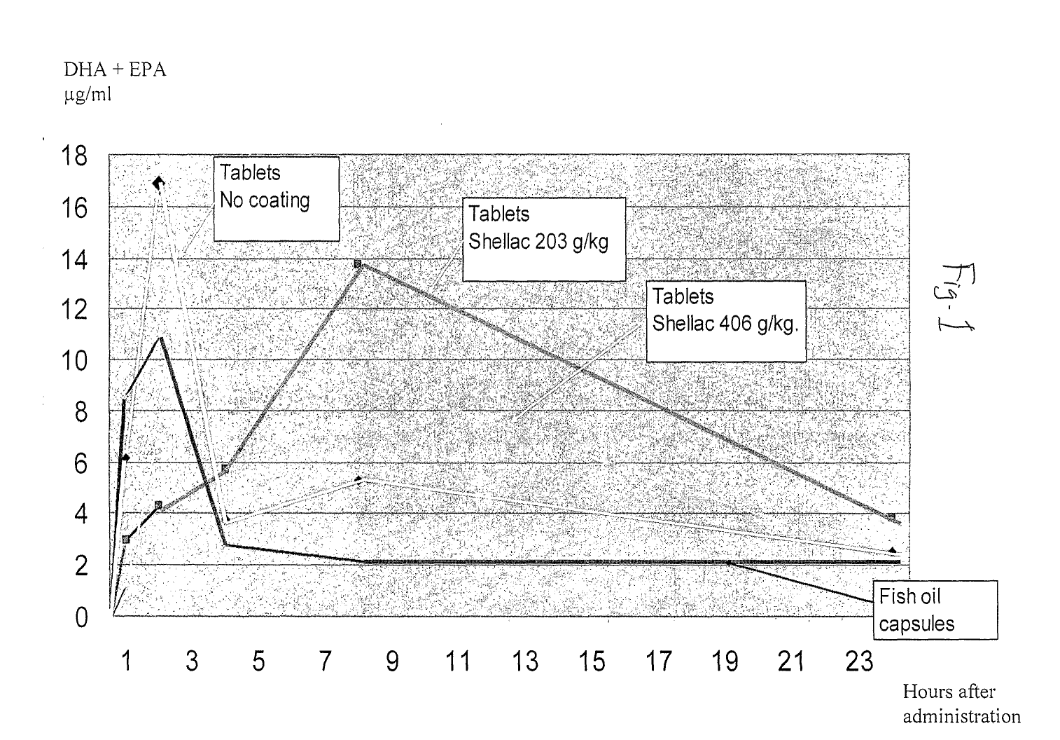

The delay in the time of release of the active substances in the tablets, in particular the fish oil was measured as the three types of tablets were tested against conventional fish oil capsules with an equivalent amount of oil. The tablets were given to minipigs and the concentration of EPA and DHA was measured at 0 and after ½, 1, 2, 4, 8, 12 and 24 hours after the intake. The results are shown in FIG. 1.

FIG. 1 shows a diagram plotting the DHA and EPA content in μg/ml in the blood against the hours after administration. In the diagram four curves are presented representing the measurements for each of the tablet types mentioned above and for the fish oil capsules.

From FIG. 1 it becomes apparent that Cmax for the concentration of DHA/EPA, the tablets are later than for capsules. This shows the prolonged release properties of the tablets. Further, AUC (Area Under Curve) is larger for the tablets than for the oil capsules, showing that the absorption of DHA/EPA for the tablets is more complete than from the capsules. Therefore, the formulation gives extended release and a more complete absorption.

1. A tablet comprising
a) a tabletted core comprising
a triglyceride granulate comprising triglycerides containing one or more esterified omega-3 fatty acids, and
excipients, and
b) an enteric coating surrounding the tabletted core, said enteric coating comprising a coating material.

2. The tablet according to claim 1, wherein the tabletted core additionally comprises one or more vitamins and/or one or more minerals

3. The tablet according to claim 1, wherein the coating is a pH-sensitive, enteric coating.

4. The tablet according to claim 1, wherein the tablet releases at most 25% of the triglyceride during the first two hours of testing when performing a dissolution release profile in simulated gastric fluid using Ph. Eur. paddle method at 100 rpm.

5. The tablet according to claim 1, wherein the tablet releases at least 10% of the triglyceride during the first two hours of testing when performing a dissolution release profile in simulated intestinal fluid using Ph. Eur. paddle method at 100 rpm.

6. The tablet according to claim 1, wherein the coating material comprises a pharmaceutically acceptable acid-resistant polymer.

7. The tablet according to claim 1, wherein the coating material has a solubility at 25° C. of at most 5 g coating material per 100 g acidic aqueous solution, at most 2 g coating material per 100 g acidic aqueous solution, at most 5 g coating material per 100 g acidic aqueous solution, at most 10 g coating material per 100 g acidic aqueous solution, or at most 15 g coating material per 100 g acidic aqueous solution, said acid aqueous solution consisting of 1 mM HCl dissolved in demineralised water.

8. The tablet according to claim 1, wherein the coating material has a solubility at 25° C. of at least 0.5 g coating material per 100 g basic aqueous solution, at least 1 g coating material per 100 g basic aqueous solution, at least 5 g coating material per 100 g basic aqueous solution, at least 10 g coating material per 100 g basic aqueous solution, or at least 15 g coating material per 100 g basic aqueous solution, said basic aqueous solution consisting of 1 mM NaOH dissolved in demineralised water.

9. The tablet according to claim 1, wherein the coating material comprises at least one material selected from the group consisting of shellac, acid-resistant acrylic polymer, acid-resistant methacrylic polymer, modified cellulose, methacrylic acid copolymers, cellulose acetate (and its succinate and phthalate version), styrol maleic acid co-polymers, polymethacrylic acid/acrylic acid copolymer, hydroxypropyl methyl cellulose phthalate, polyvinyl acetate phthalate, hydroxyethyl ethyl cellulose phthalate, hydroxypropyl methyl cellulose acetate succinate, cellulose acetate tetrahydrophtalate, acrylic resin, timellitate, shellac, and combinations thereof.

10. The tablet according to claim 1, wherein the coating material comprises one or more coating additives.

11. The tablet according to claim 1, wherein the smallest thickness of the coating is at least 5 micron, at least 20 micron, at least 50 micron at least 100 micron, at least 200 micron or at least 400 micron.

12. The tablet according to claim 1, wherein the thickness of the coating is in the range of 1 micron-5 mm, 10 micron to 1 mm, 25 micron-500 micron, 50 micron-250 micron, or 75 microns-150 micron.

13. The tablet according to claim 1, wherein the triglyceride granulate has an average granule size in the range of 5 micron-2 mm, 10 micron to 1.5 0 mm, 25 micron-1 mm, 50 micron-500 micron, or 75 microns-250 micron.

14. The tablet according to claim 1, wherein the triglyceride granulate comprises in the range of 5-80% triglyceride by weight, 10-70% triglyceride by weight, 15-60% triglyceride by weight, 20-50% triglyceride by weight, or 25-45% triglyceride by weight.

15. The tablet according to claim 1, wherein the triglyceride granulate comprises in the range of 2-75% esterified omega-3 fatty acids by weight, 5-70% esterified omega-3 fatty acids by weight, 10-60% esterified omega-3 fatty acids by weight, of 15-45% esterified omega-3 fatty acids by weight, or 20-40% esterified omega-3 fatty acids by weight.

16. The tablet according to claim 1, wherein the triglyceride granulate comprises one or more granulate additives.

17. The tablet according to claim 1, wherein the tabletted core comprises in the range of 20-85% triglyceride granulate by weight, 25-75% triglyceride granulate by weight, 30-70% triglyceride granulate by weight, 35-65% triglyceride granulate by weight, or 40-60% triglyceride granulate by weight.

18. The tablet according to claim 1, wherein the tabletted core comprises in the range of 10-70% triglyceride by weight, 15-65% triglyceride by weight, 20-60% triglyceride by weight, 25-55% triglyceride by weight, or 30-50% triglyceride by weight.

19. The tablet according to claim 1, wherein the tabletted core comprises one or more excipients.

20. The tablet according to any of the preceding claims, wherein the tablet comprises in the range of 0.1-50% esterified omega-3 fatty acids by weight, such as 0.5-25% esterified omega-3 fatty acids by weight, preferably in the range of 1-20% esterified omega-3 fatty acids by weight, and even more preferably in the range of 2-18% esterified omega-3 fatty acids by weight, such as in the range of 5-15% esterified omega-3 fatty acids by weight.

21. The tablet according to claim 1, wherein the tablet comprises in the range of 1-99% tabletted core by weight, 10-90% tabletted core by weight, 20-80% tabletted core by weight 25-75% tabletted core by weight, 30-70% tabletted core by weight, or 40-60% tabletted core by weight.

22. The tablet according to claim 1, wherein the tablet comprises in the range of 1-75% coating by weight, 10-65% coating by weight, 15-60% coating by weight 20-55% coating by weight, 25-50% coating by weight, or of 30-45% coating by weight.

23. The tablet according to claim 1, wherein the tablet has a weight in the range of 100 mg-5 g, 250 mg-2.5 g, 500 mg-2 g, or 750 mg-1.5 g.

24. A method of preparing a tablet comprising a coated, tabletted core, the method comprising the steps of
i) providing a tabletted core comprising
a triglyceride granulate comprising triglycerides containing one or more esterified omega-3 fatty acids,
a nutricient,
excipients, and
ii) coating said tabletted core with an enteric coating material.

25. The method according to claim 24, therein the coating is applied as an aqueous film of a coating solution comprising the coating material.

26. (canceled)
